(19) United States
(12) Patent Application Publication
(10) Pub. No.: US 2019/0117497 A1
(43) Pub. Date: Apr. 25, 2019

(54) SKIN CLEANSERS AND METHODS OF USE

(71) Applicant: Filip Sedic, Shanghai (CN)

(72) Inventor: Filip Sedic, Shanghai (CN)

(21) Appl. No.: 16/149,447

(22) Filed: Oct. 2, 2018

Related U.S. Application Data
(60) Provisional application No. 62/574,369, filed on Oct. 19, 2017.

(51) Int. Cl. A61H 23/00; A61B 5/00; A61B 5/053; A61H 23/02; A47K 7/02

(52) U.S. Cl. CPC A61H 23/006; A61B 5/443; A61B 5/0531; A61B 5/0537; A61H 2201/501; A47K 7/02; A61H 2201/5043; A61H 2230/655; A61H 23/0218

(57) ABSTRACT

An example skin cleanser includes an exterior covering, such as a silicone exterior covering, with at least one textured portion for transmitting vibrational tapping to the skin of a user. The skin cleanser includes at least one motor for generating vibrations that are transmitted to the skin. The textured portion includes touch-points that transmit the vibrations to the skin in contact with the textured portions. The touch-points may include thicker and thinner formations to provide firmer or softer vibrations to the skin. The skin cleanser includes at least one sensor configured to collect data relating to moisture levels of the skin. The skin cleanser also includes an interface configured to transmit information to other devices.

### CROSS REFERENCE TO RELATED APPLICATIONS

This application claims the benefit of U.S. Provisional Application No. 62/574,369, filed Oct. 19, 2017. This related application is incorporated by reference into this disclosure in its entirety.

### FIELD

The disclosure relates generally to the field of beauty-enhancing devices and methods of use. More particularly, the disclosure relates to beauty-enhancing devices suitable for use on the human face and body and various methods of use. Specific examples relate to the field of beauty-enhancing skin cleansers and methods of use.

### BACKGROUND

Skin health and appearance is an important aspect of many beauty regimens. Typical skin care focuses on particular creams or lotions to be applied to the skin, usually performed manually or by sponge or brush. Cleaning by hand often fails to adequately apply lotions to the skin, and can be ineffective at removing oils and other contaminants. An effective skin cleanser should clean the face more effectively than hand cleaning but avoid abrasions or other harsh impacts on the skin. Additionally, it would be beneficial for users of skin cleansers to be able to track their use of such devices, measure moisture levels of the skin before, during, and after use of a skin cleanser, and transmit this information to other devices for analysis.

The art includes several skin cleansers. These skin cleansers, however, do not allow for users to track their uses of the device, measure moisture levels of the skin, and/or transmit this information to other devices for analysis.

A need exists, therefore, for improved skin cleansers and methods of use.

### BRIEF SUMMARY OF SELECTED EXAMPLES

Various example skin cleansers and methods of use are described.

An example skin cleanser having a skin cleanser body and configured to be used on skin of a user comprises a substantially flat base configured to stand unaided on a substantially flat surface, said skin cleanser body having a cross-sectional shape that is longer in a first direction substantially parallel to the base than in a second direction substantially parallel to the base, a sensor disposed within said skin cleanser body, the sensor having a first sensor end and a second sensor end extending outside said skin cleanser body, the sensor configured to measure moisture level readings of said skin, a controller disposed within said skin cleanser body, the controller being capable of collecting, storing, and transmitting data related to the moisture level readings, a silicone exterior covering that covers substantially all of an exterior of said skin cleanser body, the silicone exterior covering having a first side and a second side, the first side defining a first textured surface area that is integrally formed with the silicone exterior covering, the first textured surface area comprising a first set of touch-points, the second side defining passageways through which the first sensor end and the second sensor end, a motor disposed within said skin cleanser body configured to produce pulsations of the skin cleanser, and at least one control disposed on said skin cleanser body configured to operate the motor.

Another example skin cleanser having a skin cleanser body and configured to be used on skin of a user comprises a substantially flat base configured to stand unaided on a substantially flat surface, said skin cleanser body having a cross-sectional shape that is longer in a first direction substantially parallel to the base than in a second direction substantially parallel to the base, a sensor disposed within said skin cleanser body, the sensor having a first sensor end and a second sensor end extending outside said skin cleanser body, the first sensor end and second sensor end being gold-plated, the sensor configured to measure moisture level readings of said skin, a controller disposed within said skin cleanser body, the controller being capable of collecting, storing, and transmitting data related to the moisture level readings of said skin, a silicone exterior covering that covers substantially all of an exterior of said skin cleanser body, the silicone exterior covering having a first side and a second side, the first side defining a first textured surface area that is integrally formed with the silicone exterior covering, the first textured surface area comprising a first set of touch-points, the second side defining passageways through which the first sensor end and the second sensor end extend, a motor disposed within said skin cleanser body configured to produce pulsations of the skin cleanser, and at least one control disposed on said skin cleanser body configured to operate the motor.

A method of calculating a skin age of skin of a user, comprises the steps of operatively connecting a skin cleanser with a mobile phone, the skin cleanser having a skin cleanser body and configured to be used on the skin of a user, comprising a substantially flat base configured to stand unaided on a substantially flat surface, said skin cleanser body having a cross-sectional shape that is longer in a first direction substantially parallel to the base than in a second direction substantially parallel to the base, a sensor disposed within said skin cleanser body, the sensor having a first sensor end and a second sensor end extending outside said skin cleanser body, the first sensor end and the second sensor end being gold-plated, the sensor configured to measure moisture level readings of said skin, a controller disposed within said skin cleanser body, the controller being capable of collecting, storing, and transmitting data related to the moisture level readings of said skin, a silicone exterior covering that covers substantially all of an exterior of said skin cleanser body, the silicone exterior covering having a first side and a second side, the first side defining a first textured surface area that is integrally formed with the silicone exterior covering, the first textured surface area comprising a first set of touch-points, the second side defining passageways through which the first sensor end and the second sensor end extend, a motor disposed within said skin cleanser body configured to produce pulsations of the skin cleanser, and at least one control disposed on said skin cleanser body configured to operate the motor, activating the skin cleanser such that the sensor measures moisture level readings of said skin of said user, placing the sensor in contact with said skin of said user in order to allow the sensor to measure the moisture level readings, transmitting the moisture level readings to the mobile phone, calculating said user's AMR via software stored on the mobile phone, entering a user's age into the software stored on the mobile phone, comparing the user's AMR data with said user's age, and calculating said user's skin age.

Additional understanding of claimed devices and methods may be obtained by reviewing the detailed description of selected examples, below, with reference to the appended drawings.

### DESCRIPTION OF FIGURES

FIG. 1 is a perspective view of an example skin cleanser.

FIG. 2 is an exploded view of the skin cleanser illustrated in FIG. 1.

FIG. 3 is an end view of the skin cleanser illustrated in FIG. 1.

FIG. 4 is a side view of the skin cleanser illustrated in FIG. 1.

FIG. 5 is a cross-sectional view of the skin cleanser illustrated in FIG. 3, taken along line 5-5.

FIG. 6 is a cross-sectional view of the skin cleanser illustrated in FIG. 4, taken along line 6-6.

FIG. 7 is another end view of the skin cleanser illustrated in FIG. 1.

FIG. 7A is an end view of an alternative skin cleanser.

FIG. 8 is a bottom view of the skin cleanser illustrated in FIG. 1.

FIG. 9 is a top view of the skin cleanser illustrated in FIG. 1.

FIG. 10 is a diagram illustrating components of a networked skin cleanser in an example embodiment.

FIG. 11 is a flowchart representation of an example method of verifying authenticity of a skin cleanser.

FIG. 12 is a flowchart representation of an example method of calculating a user's skin moisture type.

FIG. 13 is a flowchart representation of an example method of calculating a user's skin age.

FIG. 14 is an end view of an example system that includes a skin cleanser and a second device.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION OF SELECTED EXAMPLES

The following detailed description and the appended drawings describe and illustrate various skin cleansers and methods of use. The description and drawings are provided to enable one skilled in the art to make and use one or more example skin cleansers and methods of use. They are not intended to limit the scope of the claims in any manner.

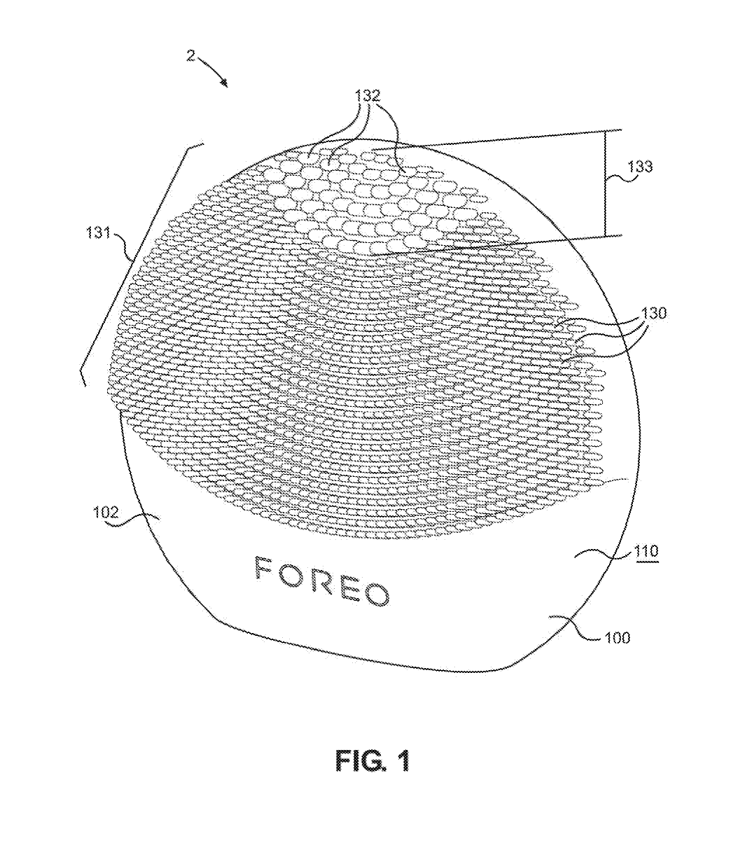

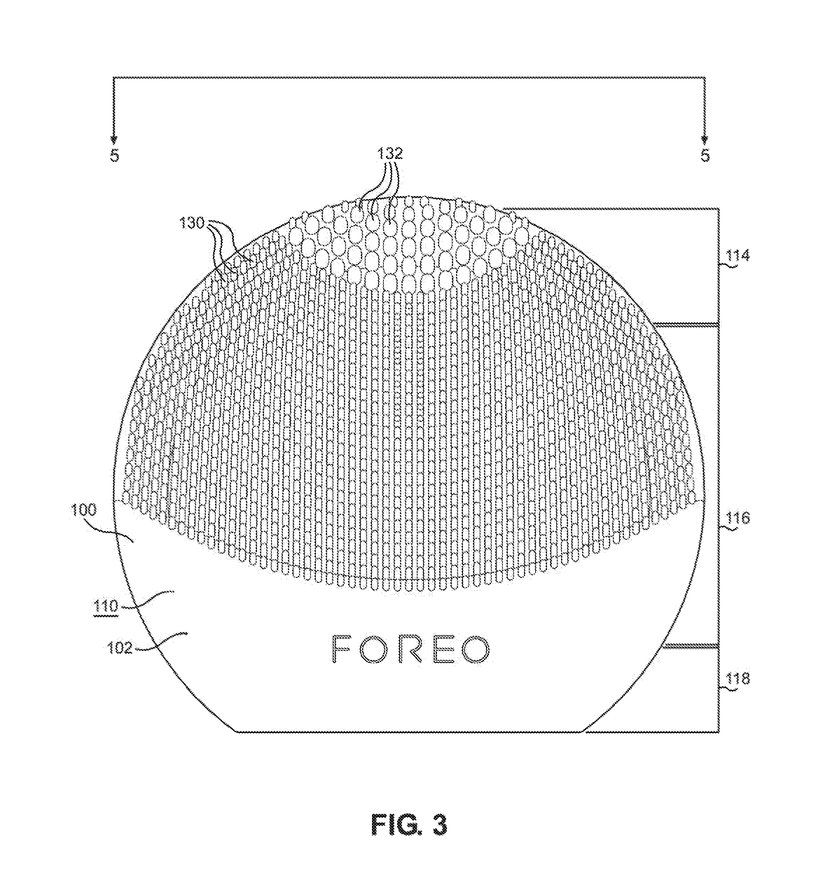

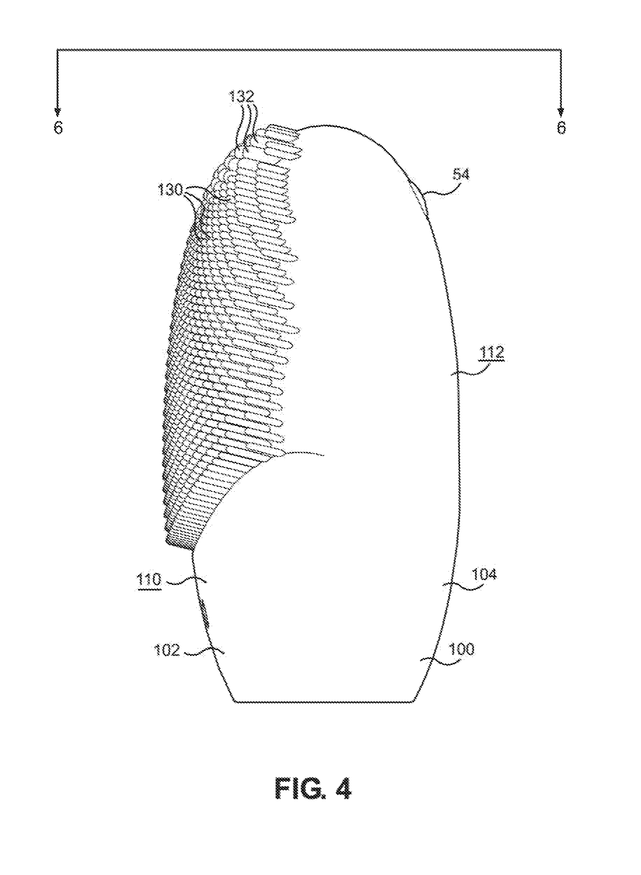

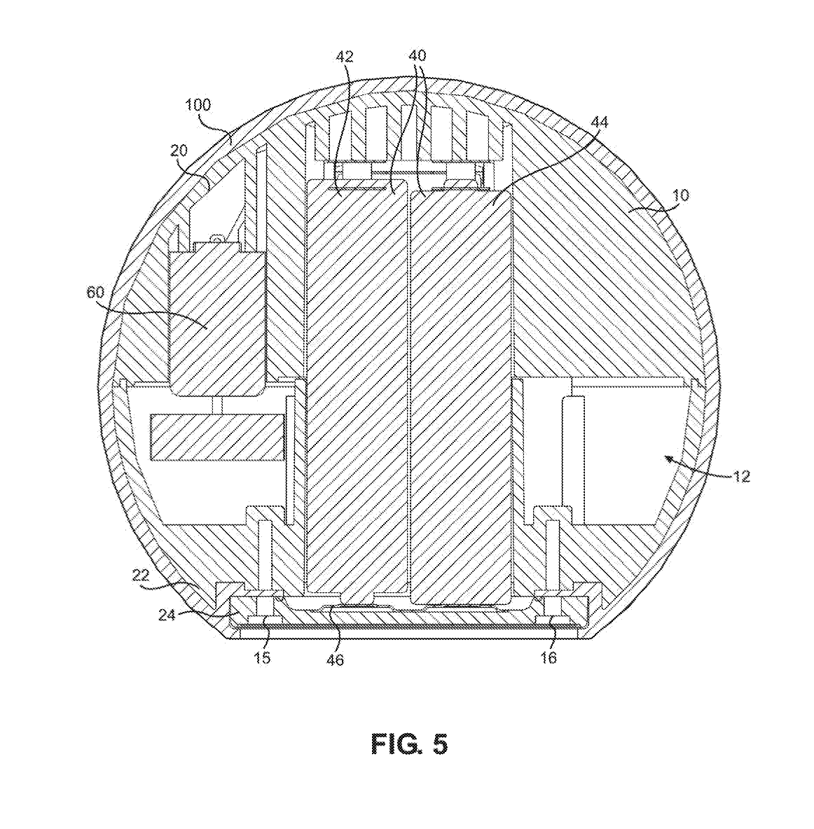

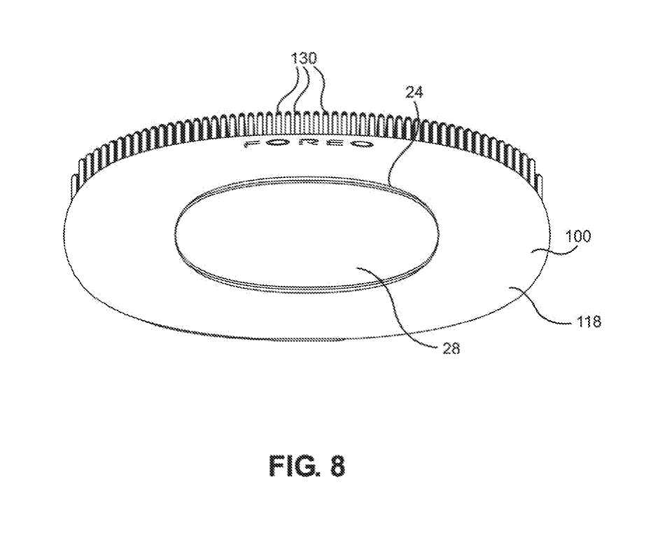

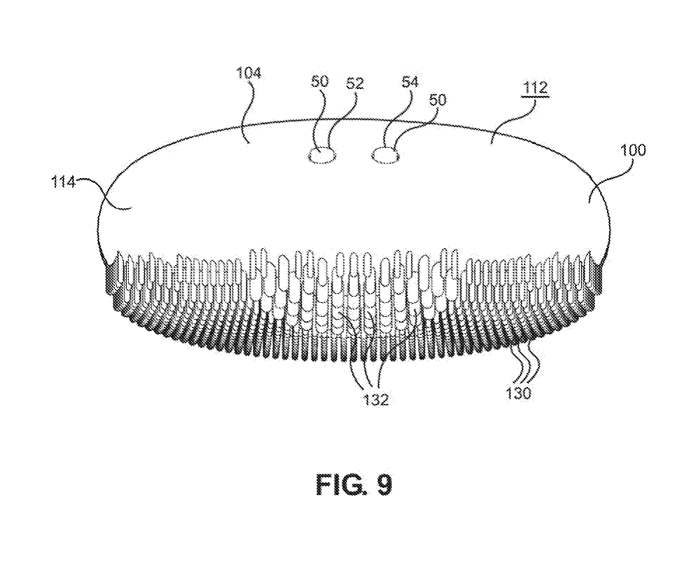

FIGS. 1, 2, 3, 4, 5, 6, 7, 8, and 9 illustrate an example skin cleanser 2. The skin cleanser comprises a main body 10 and an exterior covering 100. The main body 10 comprises at least a first piece 20, a second piece 22, and a base 24.

The first piece 20, the second piece 22, and the base 24 connect to one another to form the main body 10. The second piece 22 is disposed adjacent to and between of the first piece 20 and the base 24. The first end 21 of the first piece 20 is configured to mechanically attach to the first end 23 of the second piece 22. The first end 21 of the first piece 20 snugly and firmly attaches to the first end 23 of the second piece 22. The second end 25 of the second piece 22 is configured to mechanically attach to the first end 27 of the base 24. The second end 25 of the second piece 22 snugly and firmly attaches to the first end 27 of the base 24. The second end 29 of the base 24 is substantially flat and is configured such that the skin cleanser 2 may stand without assistance when placed on a relatively flat surface, such as a table. The main body 10 is comprised of plastic in this embodiment; however, in other embodiments, other suitable materials may comprise the main body. A skilled artisan will be able to determine how to suitably configure the first piece, the second piece, and the base according to various considerations, including the size and shape of the skin cleanser and the desired power source. In other embodiments, the skin cleanser may include one, three, or more than three pieces. In a different embodiment, the first piece, the second piece, and the base may be attached via any mechanism, including through the use of an adhesive. In an alternative embodiment, the main body may be a single, integral piece. In another embodiment, the base may not be flat, and the skin cleanser may not be able to stand unassisted on substantially flat surfaces.

Optionally, a label 28 may be affixed to the second end 29 of the base 24. The label 28 may be mechanically or adhesively attached to the second end 29 and may contain information pertaining to the skin cleanser 2 and/or the entity selling or distributing the same such as: serial number, place of manufacture, contact information of manufacturer, contact information of seller, and various other pieces of information.

The main body 10 defines a cavity 12, which houses a controller 30, a power source 40, a sensor 50, and a motor 60. The main body 10 may have any suitable shape. A skilled artisan will be able to select a suitable shape for the main body according to a particular example based on various considerations, including the strength of the motor and facial shapes of prospective users. In various embodiments, the skin cleanser main body can be configured to have different shapes, such as a substantially oval shape, a substantially round shape, a triangular shape, a substantially leaf-like shape, a curved shape, and so forth. The oval or round shape of the body allows the user to hold the cleanser in the palm of the hand, possibly with fingers splayed along the back side of the cleanser and thumb against the controls in the front side of the skin cleanser. In some embodiments, the cleanser is wider than it is thick, for example. This configuration allows the user to easily hold the cleanser in the palm of the hand and reach his or her fingers around both side of the cleanser for easy and ergonomic manipulation of the cleanser against the skin.

The motor 60 is enclosed in a frame (not illustrated in the Figures) within the main body 10 and is disposed within the cavity 12. In some embodiments, there is more than one motor; in embodiments containing multiple motors, the motors may vary from one another in frequency output. The controller 30, provides instructions to the motor 60, which is powered by the power source 40. A high- or low-frequency motor may be used to create pulsations that vibrate the skin cleanser in various embodiments. The motor 60 in this embodiment provides vibration frequencies between about 50 hertz (“Hz”) and about 300 Hz, though pulsation frequencies higher and lower than this range may also provide different types of skin cleansing. When the skin cleanser 2, and the exterior covering 100 in particular, is applied to the body, such as the face or neck, the pulsations provide a thorough cleaning of the skin. A skilled artisan will be able to select suitable motors and vibration ranges based on various considerations, including the size and shape of the skin cleanser and the desired vibration strength of the device. Examples of suitable ranges for vibration frequencies include vibration frequencies between about 20 Hz and about 500 Hz, vibration frequencies between about 100 Hz and about 400 Hz, and vibration frequencies between about 200 Hz and about 300 Hz. In another embodiment, the skin cleanser may comprise two or more motors. In another embodiment, the motor may be stochastic.

The pulsations provide a tapping motion to the skin, in some embodiments, by providing impulses to the skin's surface from the silicone touch-points (described below) pulsating against the skin's surface. The tapping-based cleansing provided by such a skin cleanser provides a deeper clean that is less abrasive than scrubbing the skin with harsher bristles of other materials (e.g., nylon brush bristles). Frequencies in this range provide deep facial cleansing of oil and dirt, unclog follicles, and stimulate blood circulation and lymph flow within the skin. Additional benefits can range from stimulating collagen synthesis in fibroblast cells to improving the flow of blood and lymphatic fluid—the result is healthier, younger-looking skin, such as in the known problem areas of the face, and a more sharply defined profile of the face. In particular, when gently applied to expression-line problem areas at the brow, temples and nasolabial folds, the skin cleanser can relax underlying muscles and reduce the appearance of dynamic wrinkles when such frequencies are used.

The power source 40 is also disposed within the cavity 12 of the main body 10. The power source 40 provides power to each of the controller 30, the sensor 50, and the motor 60. In the illustrated embodiment, the power source 40 comprises a first battery 42 and a second battery 44 (collectively, the “batteries 42, 44”) which extend lengthwise adjacent the controller 30 within the skin cleanser 2. The batteries 42, 44 are replaceable, dry cell batteries in the illustrated embodiment. The base 24 may be removed from the rest of the main body 10 through the removal of a first connection mechanism 15 and a second connection mechanism 16, enabling a user to replace the batteries 42, 44 as needed. The first 15 and second 16 connection mechanisms comprise screws in the illustrated embodiment. The batteries 42, 44 are attached to a battery bracket 46 disposed within the cavity 12, as well, which stabilizes the batteries within the main body 10. A skilled artisan will be able to determine a suitable power source according to a particular example based on various configurations, including the size and strength of the motor and the size of the main body of the skin cleanser. In other embodiments, the power source may comprise one, two, three, or more than three batteries. In various examples, the batteries may be Single A, Double A, Triple A, or any other suitable type of battery. In an alternative example, the battery may be rechargeable battery (such as a lithium-ion battery) and may be charged through the use of a charging port, such as a DC jack. In various other embodiments, any suitable physical mechanism (and zero, one, three, or more than three suitable physical mechanisms) may comprise the connection mechanisms.

The sensor 50 is also disposed within the cavity 12 of the main body 10. The sensor includes a first sensor end 52 and a second sensor end 54. The first and second sensor ends 52, 54 cooperatively work to sense the moisture level of skin which the sensor 50 is in contact with. The sensor 50 is operatively connected and attached to the controller 30 in the illustrated embodiment. The first sensor end 52 protrudes from a first passageway (not illustrated in the Figures) defined by the rear side 19 of the first piece 20 of the main body 10 and a first passageway (not illustrated in the Figures) defined by the second exterior covering side 104 such that a user is able to contact the first sensor end 52. The second sensor end 54 protrudes from a second passageway (not illustrated in the Figures) defined by the rear side 19 of the first piece 20 of the main body 10 and a second passageway (not illustrated in the Figures) defined by the second exterior covering side 104 such that a user is able to contact the second sensor end 54. Optionally, each of the first sensor end 52 and the second sensor end 54 may be plated with a material to prevent discoloration of the skin of the user based on repeated contact with the sensor 50. The first sensor end 52 and the second sensor end 54 are gold-plated in the illustrated embodiment. Additionally, in various embodiments, any suitable sensor may be used, including a capacitive sensor. A skilled artisan will be able to select a suitable sensor and structures to ensure that the sensor ends contact the user according to a particular example based on various considerations, including the data to be collected by the sensor and the shape of the skin cleanser main body. In different embodiments, the sensor may comprise a battery sensor. In other embodiments, the skin cleanser may comprise zero, two, three, or more than three sensors. In various embodiments, all or a portion of the sensor(s) may be coated with one or more additional or different materials, including nickel and/or platinum. In alternate embodiments, one or more of the sensor(s) may be disposed at any suitable location on the skin cleanser, so long as a user will be able to contact said sensor(s). In still other embodiments, the sensor may be operatively connected to the controller in any way, including via a physical mechanism or through the use of an adhesive.

The described structure allows for a user to contact the sensor 50 so that it may collect data pertaining to the moisture level of the skin of a user. The sensor 50 is able to measure said moisture level by monitoring the resistance and a related voltage differential sensed by the first sensor end 52 and the second sensor end 54 when the sensor 50 is in contact with the skin; the sensor 50 then communicates this value to the controller 30. As described below, the controller 30 can then transmit this information to one or more devices for analysis.

In an alternative embodiment, a sensor is also included in the skin cleanser near the front or back of the skin cleanser (or both). The sensor may be a pressure sensor, or a similar component, that detects a user's action to activate the sensor, such as via a user pressing the device to his or her body. In one embodiment, the controller activates at least one of the motors when the sensor is activated, permitting the device to automatically activate when the user activates the sensor. Multiple sensors may be included to activate different functions. For instance, in one configuration a sensor is located underneath a textured portion of the skin cleanser and detects a user's contact with that textured portion. Based on which sensor is activated, the controller activates an operational mode suitable for the side on which the sensor was activated. For example, activating the motor at a low frequency when one side is contacted, and activating the motor at a high frequency when the other side is contacted.

The main body 10 also houses a controller 30, which is disposed within the main body 10 such that it extends lengthwise along the main body 10. The controller 30 is operatively connected to the sensor 50, and the motor 60 and provides instructions to and controls each of these components. The controller 30 comprises a printed circuit board assembly (“PCBA”) and related circuitry; however, in other embodiments, the controller may comprise any device suitable to control the skin cleanser's components, such as a printed circuit board (“PCB”) or similar component. Additionally, the controller 30 is operatively connected to an interface 210 which allows it to communicate with a second device (described in greater detail, below).

The main body 10 also includes a user control 70 that allows a user to operate the skin cleanser 2. The user control 70 is disposed on the rear of the main body 10 and is operatively connected to the controller 30. The user control 70 comprises a button in the illustrated embodiment, which allows for a user to active the skin cleanser 2 by pressing the button. Optionally, repeated activation of the user control 70 allows for a user to cycle through various modes of operation of the skin cleanser 2. Potential modes that may be activated by the user control 70 include low frequency mode, high frequency mode, stochastic mode, and alternating frequency mode. A skilled artisan will be able to suitably place the user control on the device and configure the same according to a particular example based on various considerations, including the number of desired modes and the desired placement of the touch-points of the silicone exterior covering. In other embodiments, the user control may be disposed on the front of the main body, the side(s) of the main body, or the base of the main body. In a different embodiment, the skin cleanser may comprise zero, one, three, or more than three user controls. In additional embodiments, the user control may include a first user control to increase frequency of the motor and a second user control to decrease frequency of the motor.

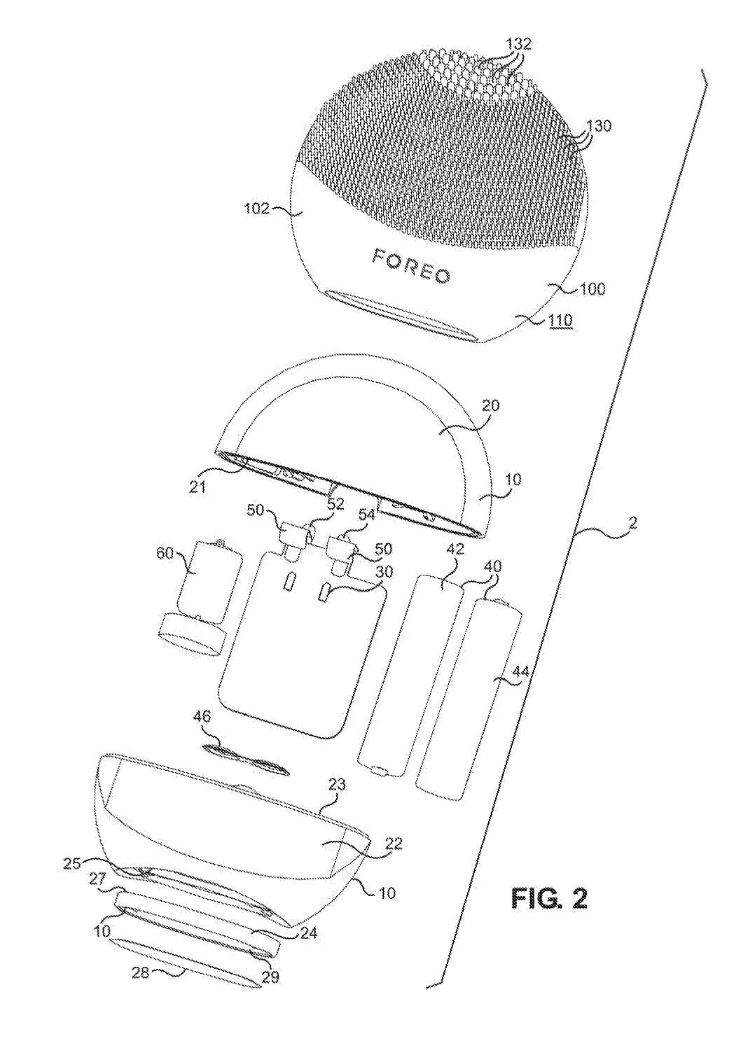

FIG. 2 best illustrates the exterior covering 100 when it is separate from the main body 10. Various other FIGS., including FIGS. 1, 3, 4, 7, 8, and 9 clearly illustrate the exterior covering 100 once it has been placed about the main body 10. The exterior covering 100 comprises a single, unitary piece in this embodiment. The exterior covering 100 can be comprised of various elastic materials, preferably materials that are soft and do not damage the skin, such as silicone. Silicone is used as an example throughout, though it is understood that other materials can be used, as well. The exterior covering 100 is made of a hygienic silicone that is fast-drying and non-absorbent, allowing the skin cleanser 2 to be used with many skin cleaning products without wear. The exterior covering 100 includes a front surface 110 on a first exterior covering side 102 and a back surface 112 on a second exterior covering side 104 having varying textures. The exterior covering 100 also includes an upper portion 114, a middle portion 116, and a lower portion 118 and may define various structures on said front and back 110, 112, surfaces. The exterior covering 100 also defines first and second passageways (not illustrated in the Figures) through which the first sensor end 52 and the second sensor end 54 extend such that a user is able to contact the first sensor end 52 and second sensor end 54.

For example, the front surface 110 comprises a series of thinner touch-points 130, (grouped in a second textured surface area 133) for gentle cleansing of non-oily or sensitive skin, with an area of thicker touch-points 132 (grouped in a first textured surface area 131) disposed towards the upper portion 114 of the exterior covering 100. Providing more resistance than the thinner touch-points 130, the thicker touch-points 132 allow for more targeted cleansing of oilier areas and hard to-reach points around the nose, ears and hairline. The pattern of touch-points presented in the Figures is one example of a pattern that might be used to cleansing of what is commonly referred to as “normal” skin. Normal skin can include some areas that are drier, oilier, or more sensitive, such that the different patterns of thinner touch-points 130 and thicker touch-points 132 (collectively, the “touch-points 130, 132”) can be helpful in targeting these areas (e.g., thinner touch-points for oily areas around the nose). The thinner touch-points 130 and thicker touch-points 132 may vary in size and spacing. Thus, the thinner touch-points 130 and thicker touch-points 132 can also be arranged differently about the front surface 110 than is illustrated. Additionally, the thinner and/or thicker touch-points 130, 132 and/or the main body 10 of the skin cleanser 2 itself may be compressible and bendable, such that the thinner and thicker 130, 132 touch-points and/or main body 110 of the skin cleanser 2 conform to the surface of the skin during use. A skilled artisan will be able to determine how to suitably shape, size, and place the various touch-points according to a particular example based on various considerations, including the skin type of potential users and the size and shape of the skin cleanser. In various embodiments, the thinner touch-points may be between about 25% and about 80% thinner (e.g., 30%, 40%, 50%, 60%, 70%, etc. or values in between) than the thicker touch-points. In various embodiments, the thinner touch-points may be disposed closer together (i.e., the distance between touch-points), such as between about 15% and about 60% closer to one another, as compared to the thicker touch-points. Thus, the thinner and thicker touch-points for the normal skin cleansing can also be arranged differently around the brush surface than is illustrated.

In various embodiments, the thinner and/or thicker touch-points may be between about 0.5 millimeters (“mm”) in diameter and about 4.5 mm in diameter, between about 1.5 mm in diameter and about 3.5 mm in diameter, and between about 2 mm in diameter and about 3 mm in diameter. The textured portion of the exterior covering can comprise between about 5% and about 80% of the exterior covering, between about 20% and about 60% of the exterior covering, and between about 30% and about 50% of the device according to various embodiments. The textured portion can be positioned on an upper portion or tip of the exterior covering, such as is illustrated in the Figures, but can also be otherwise positioned (e.g., at the sides, in the middle, at the bottom of the skin cleanser). In one embodiment, one or more portions of the exterior surface, e.g., the textured surfaces, are deformable or bendable. For example, where the textured surface is composed of silicone, the surface can compress or bend when pressed against the skin to mold to the surface of the skin for providing a deeper cleansing and for better cleaning of skin surfaces that are curved. In other embodiments, the textured surface can be designed to pivot relative to the brush or to include one or more portions that pivot such that the textured surface can mold to the shape of the face. In some embodiments, the exterior covering may be comprised of many materials, including any suitable elastomeric material. In different embodiments, the exterior covering can be made of multiple pieces, rather than a single unitary piece.

Furthermore, the Figures provide just one example of how the thinner 130 and thicker 132 touch-points on the exterior covering 100 can be arranged. In other embodiments, a variety of other arrangements are also possible (e.g., thinner touch-points at the top and thicker at the bottom, thinner on one side and thicker on the other side, alternating rows of thinner and thicker, various areas or groupings of thinner and thicker in different locations on the brush, and so forth). In addition, different types of touch-points can be included, such as taller or shorter touch-points, touch-points with more or less bulbous ends, touch-points with ends of different shapes (e.g., pointed, feathered, ridged, etc.), and so forth. Similarly, the touch-points can be arranged more or less densely, can be positioned on both the front and back of the brush, can be otherwise located on the brush (e.g., only in the middle, only at the edges, etc.), among other variations. The above are just some examples of different brush surface configurations matched to different skin types. Other touch-point alignments for other skin types are also possible, such as a particular pattern for dry skin, for aging skin, for combination skin or T-zone skin (e.g., oilier around the forehead, nose, and chin), among others. In some embodiments, the front textured surface includes at least two different types of touch-points (e.g., thicker and thinner). The touch-points of a type can be grouped to provide a pattern. Various patterns can correspond to a particular skin type (e.g., male, sensitive, oily, normal, etc.).

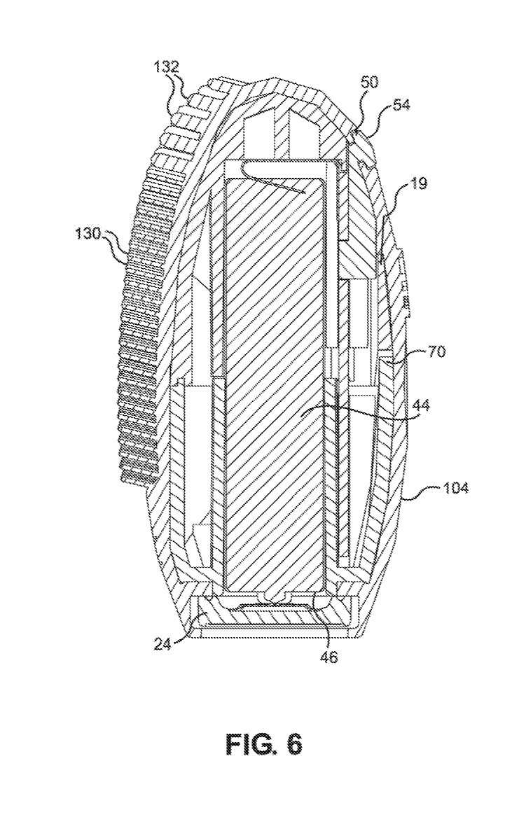

The second exterior covering side 104 includes a symbol 139 disposed adjacent the user control 70 (best illustrated in FIG. 6) in the illustrated embodiment. The symbol 139 is substantially aligned with the user control 170 so that a user may recognize that activation of the skin cleanser 2 occurs after pressing the symbol 139 disposed on the exterior covering 100 (and, thus, the user control 70). It is in the shape of a “power” sign in this embodiment. In other embodiments, the symbol may have any shape and/or size; alternatively, the exterior covering may not include a symbol. In different embodiments, the second exterior covering side comprises a series of smooth silicone ridges arranged in a wave formation, intended to provide minimal abrasion and maximize the pulsation energy transfer, and the effectiveness of the low-frequency, muscle-relaxing functions. In other embodiments, the second exterior covering side may include one or more touch-points. Thus, in such embodiments, both surfaces of the exterior covering can be used for skin cleansing.

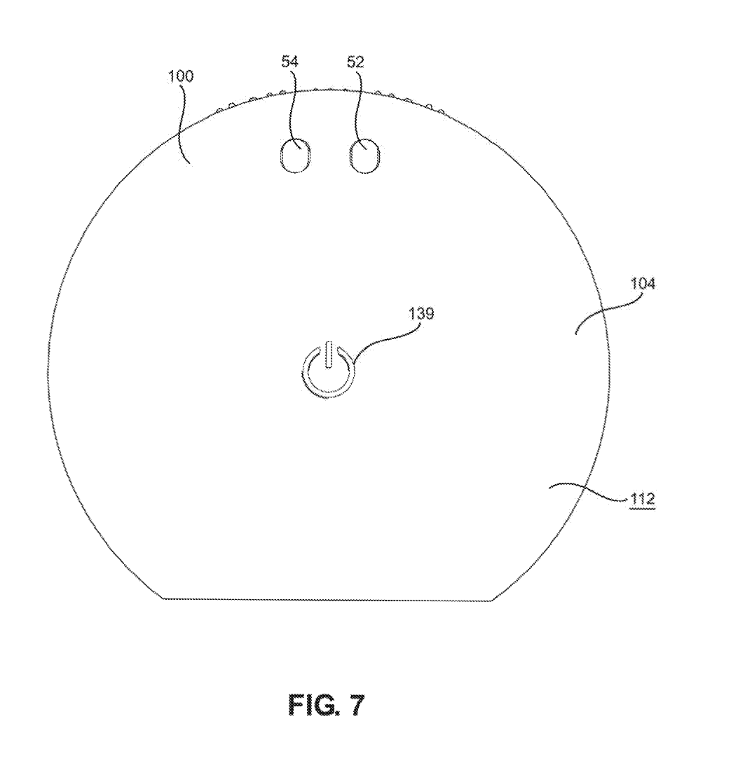

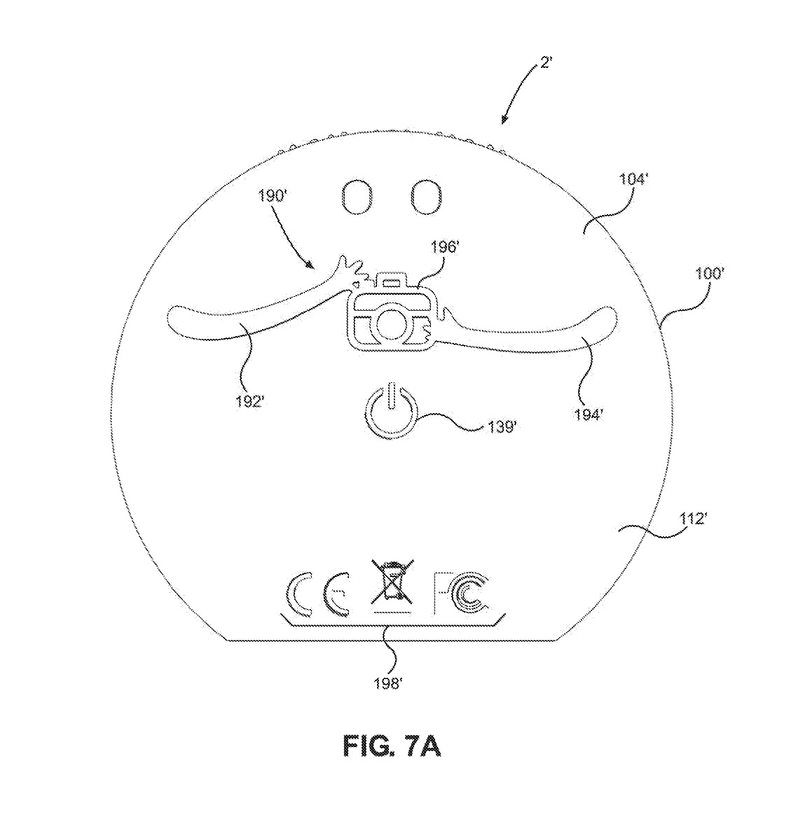

In an alternative skin cleanser 2′, illustrated in FIG. 7A, the exterior covering 100′ may include a decorative design 190′ disposed on its second exterior covering side 104′. More specifically, the decorative design 190′ may be disposed on the back surface 112′ and adjacent the symbol 139′. In the illustrated embodiment, the decorative design 190′ comprises a first structure 192′, a second structure 194′, and a third structure 196′. Each of the first 192′ and second 194′ structures are shaped and configured to resemble arms with hands. The third structure 196′ resembles a camera. The decorative design 190′ emits LED light in this embodiment when it is activated. Additionally, in this alternative, the back surface 112′ may also include one or more informational symbols 198′ designed to inform a user of a particular aspect of the product. In such an alternative, any design may be used in place of the decorative design illustrated in FIG. 7A and any informational symbol(s) may be used in place of the informational symbols illustrated in FIG. 7A.

The exterior covering 100 is designed to efficiently channel the high-frequency vibrations into the skin to cleanse deeply, unblock follicles, and to boost circulation and lymph flow. By avoiding abrasive exfoliation (as used in other brush-type devices), it remains gentle enough to use for twice-daily facial cleansing or more uses in the day, as needed. Relative to other cleansing devices, this skin cleanser 2 does not rely on a spinning or twisting action; the result is a deeper clean without the need for such abrasive scrubbing. The pulsations in combination with the first 131 and second 133 textured surface areas, the elastic, and soft material thinner 130 and thicker 132 touch-points provide thorough cleansing, since the first and second textured surface areas 131, 133 direct the power of the high-frequency motor substantially orthogonal to the skin's surface, which unlocks the skin's natural potential.

In an example embodiment, the touch-points of the textured surface areas vertically oscillate from the brush to the skin to create a tapping motion on the skin, similar to the tapping of fingertips on the skin or the patting with a towel or cotton pad. The vertical tapping of the skin in this embodiment provides a gentle cleansing of the skin, as opposed to a rotating motion that can cause a less favorable twisting or stretching of the skin that may cause damage to or scratching of the skin. The vertical tapping motion can be generated by the vibrations of the motor or of multiple motors, by other electromagnetic device(s) in the brush, or by electromechanical mechanisms, among other means. For example, the motors can be positioned in the brush to cause the vertical oscillations of the touch-points, such as by positioning one or more motors directly under or adjacent to the textured surface areas of the brush. The oscillation of the motor(s) can cause each of or at least some of the touch-points to move orthogonal to the skin's surface to tap the skin. Multiple motors can be arranged near the textured surface areas to create different motions or different speeds of vertical oscillations. For example, the motors can be positioned so that different touch-point arrangements or patterns on the textured surface areas can oscillate differently from one another to provide one type of tapping motion for some touch-points and a different type (e.g., different speed, pattern, etc.) for other touch-points. In some embodiments, each touch-point is a single structure rather than a plurality of structures, such as might be found in a brush where each brush bristle is made up of multiple bristle components arranged as a bunch.

As described above, the controller 30 also is operatively connected to an interface 210 that allows the skin cleanser 2 to communicate with a second device, such as a personal computer, tablet, mobile telephone, or other electronic device (not illustrated in the Figures). Using the interface 210, the skin cleanser 2 can send information to other devices so that the other device(s) may collect data pertaining to the use of the skin cleanser 2. Additionally, in some embodiments, the skin cleanser may receive control signals from another device that can indicate that the skin cleanser should turn on or off, increase or decrease speed, switch to a different vibration pattern, and/or switch to a particular pattern desired by the user or recommended by the other device, among other instructions. The interface 210 can be a wired or wireless interface, such as a wireless transceiver that transmits control signals between the skin cleanser 2 and a second device. A skilled artisan will be able to select a suitable interface based on various considerations, including the device with which the skin cleanser will communicate and the size and shape of the main body. In some embodiments, the interface is a radio-frequency (“RF”) transceiver used to transmit and receive RF signals between the skin cleanser and other devices. One example of an RF transceiver that could be used is a low power 2.4 GHz RF transceiver. In various embodiments, the skin cleanser may also include antennas for transmitting and receiving signals between the skin cleanser and other devices. In such examples, the interface can use BLUETOOTH®, Wi-Fi, infrared, laser light, visible light, acoustic energy, or one of a number of other methods to transmit information wirelessly between the skin cleanser and another device.

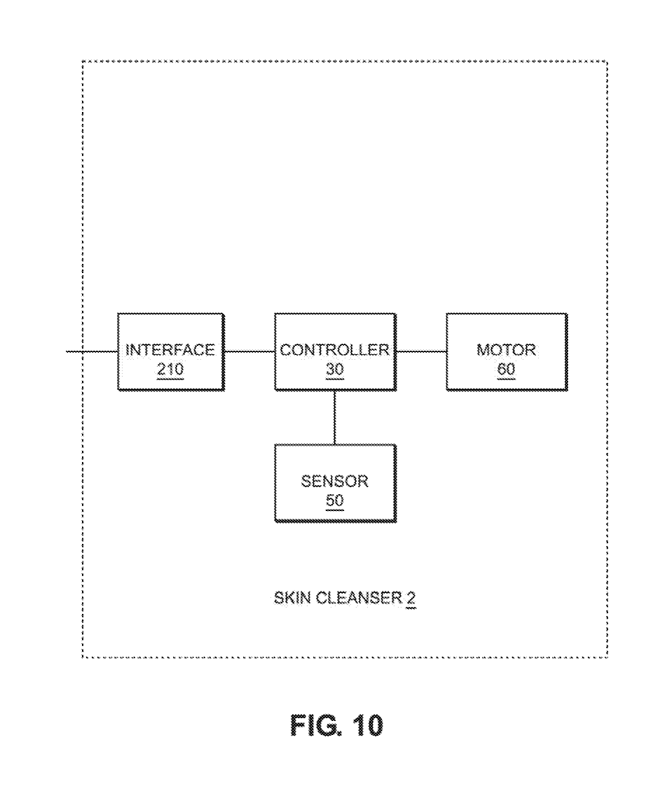

In some embodiments, the skin cleanser is connected to a network via the second device. In other embodiments, the skin cleanser is directly connected to a wireless router or cellular phone network and may connect with the second device in any of said manners. Accordingly, the skin cleanser can be controlled via personal computer, tablet, mobile phone, or other suitable electronic devices a user using the personal computer, tablet, phone, or other device. FIG. 10 illustrates one example of such a design.

FIG. 10 is a diagram illustrating component of a networked skin cleanser, such as skin cleanser 2, in accordance with an example embodiment. In this embodiment, the skin cleanser includes a controller, such as controller 30, a motor, such as motor 60, a sensor, such as sensor 50, and an interface, such as interface 210. As explained above, the skin cleanser can be connected to a network via a personal computer, tablet, mobile telephone, or other electronic device or can be directly connected to a wireless router or cellular phone network. Thus, the skin cleanser can be controlled by, transmit data to, and/or receive data from the personal computer, tablet, mobile telephone, or other electronic device via the aforementioned mechanisms. The interface may be wired or wireless and may include any of those described above. A skilled artisan will be able to determine how to suitably connect the skin cleanser with other devices based on various considerations, including the desirability of doing so and the devices to which connection would be beneficial. In some embodiments, the skin cleanser may not include an interface and, thus, may not communicate with other devices. In different embodiments, the skin cleanser may only transmit data to other devices; it may not receive any data and cannot be controlled via said other devices in this embodiment.

Examples of data that the skin cleanser may communicate to one or more of a personal computer, tablet, mobile telephone, or other electronic device may include the moisture level of a user's skin, the relative skin age of a user's skin, the elasticity of a user's skin, the presence or absence of wrinkles on a user's skin, and/or the presence and/or absence of acne or other similar skin conditions on a user's skin.

In use, a user interacts with the controller 30 through the user control 70. When activated by the user control 70, the controller 30 initiates the vibration of the motor 60 and the activation of the sensor 50. In some embodiments, the user may increase and decrease the frequency of vibration of the motor through the controller to set the frequency desired by the user. In such instances, the frequency set by the user may be stored by the controller when the controller is deactivated, such that the next time the controller is turned on the controller resumes the desired frequency.

Various other modes of use also exist. In an example embodiment, the skin cleanser may also activate a mode that directs the user to cycle through portions of the face when applying vibrations through the skin cleanser. The base of the cleanser may light up or otherwise indicate a prompt for the user to move to another portion of the face. As an example, in the mode that directs cycling through the face, the cleanser may first indicate that the user should apply the skin cleanser to the area around the cheeks for a period of time, and then may indicate that the user should apply the skin cleanser to the area around the chin and so forth until the user has cycled throughout the face. The skin cleanser can indicate when it is time to switch by, for example, lighting up the base, blinking the light a certain number of times, pulsating with a specified frequency or duration, or otherwise using vibration or light to indicate instructions to the user. Alternatively, a decorative design could emit light to indicate it is time for a user to change placement of the device on the face. Different cleaning regimens can be used for different cleansers. For example, a sensitive skin cleanser might have a cycle that is shorter in certain areas of the face to avoid irritating the face. A user might also have a program designed specifically for the user's own skin, with shorter or longer application of the brush to different areas of the face as needed for that user's skin. The program designed for a user's skin may be assessed by the manufacturer and programmed to the controller according to a diagnostic of the user's particular skincare.

As described above, the controller 30 is able to transmit data from the skin cleanser 2 (and through the interface 210) to other devices, such as a personal computer, tablet, or mobile phone (not illustrated in the Figures). Said other devices can then analyze the data sent by the skin cleanser 2 and indicate to the user how to optimally use the skin cleanser 2 in the future. Data such as skin moisture levels, number of uses, duration of uses, and intensity of use may be collected.

Various examples of data collected by the skin cleanser 2 and transmitted to another device are described herein. In one embodiment, the sensor of the skin cleanser is used to touch or be placed in closer proximity to four unique portions of the face to determine the average moisture levels of said portions. In such an embodiment, the sensor measures the surface resistance of the skin and determines a difference in voltage levels between the skin and a component of the skin cleanser. More specifically, in this embodiment the controller includes a first resistor having a reference voltage and a second resistor that receives skin surface resistance measurements from the sensor. The resistance measurements of the second resistor will vary depending on the user, time of use, particular skin qualities of a user (or as per numerous other variables), and the value received by the second resistor will cause a particular voltage decrease in the first resistor. Thus, the sensor and controller cooperatively monitor this voltage decrease and transmit this data to the software application stored on a second device such as a mobile phone. Performing this action once provides a single moisture level reading. The application receives this data after the voltage differential has been measured; thus, the unit of measurement it receives data in is the volt. The software application may either use the voltage differential data directly or input it into a formula to calculate the various measurements, described below. As is also described below, this data is used to determine and/or calculate Average Moisture Reading, skin age, skin score, and other similar pieces of information displayed via the second device. The difference in voltage between the first and second resistors comprises an individual moisture reading, which may have a value between 0 volts and 3 volts; multiple such readings comprise the Average Moisture Reading.

In such an example, the skin cleanser (and, specifically, the sensor) is placed on the right cheek, left cheek, forehead, chin, and/or nose for some period of time to determine said average moisture levels of those portions over some period of time. In various examples, the sensor may be placed in contact with the right cheek, left cheek, forehead, chin, and/or nose for between about one second and about thirty seconds, between about three seconds and about twenty seconds, and between about five seconds and about ten seconds to generate moisture readings in the manner described in the preceding paragraph. Optionally, obtaining such measurements may be repeated in order to more accurately determine moisture levels of a user. Such measurements may be repeated two, three, four, or more than four times in various embodiments. After such measurements have been taken, they will then be transmitted to a second device for analysis, as described above; for purposes of this example, the second device shall comprise a mobile phone (though other devices such as computers, tablets, or smart watches may instead be used). The device will then calculate an Average Moisture Reading (“AMR”) using all of the data collected above via dividing the sum of the various individual moisture level readings (which are measured in volts and calculated as described in the preceding paragraph) by the total number of moisture level readings performed, which will be used as described below.

In this example, the AMR is used to calculate a user's skin moisture type, skin age, and skin score. AMR can have a value between 0 volts and 3 volts. In various other examples, however, AMR may be calculated in some other manner or may have a range that extends below 0 volts and/or above 3 volts. When displayed to a user, though, the AMR will not include volts as a unit; it will only provide a reading of the AMR after it has been calculated. Thus, for clarity's sake, the unit of measurement “volts” has been omitted through the following paragraphs of the specification so that what is described will mirror what a user actually sees on the display of a second device (i.e., an AMR of 1.5, rather than 1.5 volts). Nevertheless, the various AMRs referenced below are measured in volts. In other embodiments, AMR may be calculated based on resistance measurements (and may be in ohms) or based on any other unit of measurement.

The AMR is directly used to calculate a user's moisture type. A user having an AMR that is greater than 0 and less than or equal to 1.1 indicates that a user's skin has a low moisture level. An AMR that is greater than 1.1 and less than or equal to 1.35 indicates that a user's skin has a normal moisture level. An AMR that is greater than 1.35 and up to or equal to 3 indicates that a user's skin has a high moisture level. This information will be presented to a user on the display of the second device, such as a mobile phone. The user can then use this information to modify his or her facial treatment patterns to increase the moisture levels of his or her skin. In various examples, the AMR may be used in a different manner to calculate moisture type. In some examples a user having an AMR of between about 0 and about 2, between about 0 and about 1, or between about 0 and about 0.5 may have a low moisture level. In other examples, a user having an AMR of between about 0.5 and about 2, between about 1 and about 1.5, or between about 1.2 and about 1.3 may have a normal moisture level. In additional examples a user having an AMR of between about 1 and about 3, between about 1.3 and about 3, or between about 1.35 and about 2.7 may have a high moisture level.

The AMR is also used to calculate a user's skin age. A user will first enter his or her age into a program stored on a second device, such as via a software application (or “app”) on a mobile device. Once the application has received and stored the user's age, the AMR shall be used in conjunction with the user's age to determine the user's skin age, which may differ from the actual age of the user. For example, if the user's AMR is greater than 0 and less than or equal to 1.05, the user's skin age will be the user's age plus two. Accordingly, for example, a user that is 50 years old with an AMR of 0.9 will have a skin age of 52. If the user's AMR is greater than 1.05 and less than or equal to 1.1, the user's skin age will be the user's age plus one. Accordingly, for example, a user that is 50 years old with an AMR of 1.07 will have a skin age of 51. If the user's AMR is greater than 1.1 and less than or equal to 1.35, the user's skin age will be the user's age. Accordingly, for example, a user that is 50 years old with an AMR of 1.22 will have a skin age of 50. If the user's AMR is greater than 1.35 and less than or equal to 1.6, the user's skin age will be the user's age minus one. Accordingly, for example, a user that is 50 years old with an AMR of 1.42 will have a skin age of 49. If the user's AMR is greater than 1.6 and less than or equal to 3, the user's skin age will be the user's age minus two. Accordingly, for example, a user that is 50 years old with an AMR of 1.84 will have a skin age of 48. A skilled artisan will be able to determine how best to use AMR and a user's age to calculate skin age according to a particular example based on various considerations, including the characteristics of the anticipated user, the sensitivity of the sensors used, and the anticipated moisture of an area in which users reside. In various other examples, zero, one, two, three, four, or more than four years may be subtracted from a user's age based on various AMRs to determine skin age. In other examples, zero, one, two, three, four, or more than four years may be added to a user's age based on various AMRs.

The AMR may also be used to calculate a user's skin score in various embodiments. In one such embodiment, skin scores may range from 50 to 100 points and be comprised by combining values set by software stored on an application of a second device, such as a mobile phone. The skin score may be calculated by assigning the AMR a certain point value and assigning days since the user's first use of the skin cleanser a point value and combining the same. In said embodiment, for example, an AMR of less than or equal to 1.05 will contribute 50 points to a user's skin score; an AMR greater than 1.05 and less than or equal to 1.1 will contribute 52.5 points to a user's skin score; an AMR greater than 1.1 and less than or equal to 1.35 will contribute 55 points to a user's skin score; an AMR greater than 1.35 and less than or equal to 1.6 will contribute 57.5 points to a user's skin score; and an AMR of greater than 1.6 will contribute 60 points to a user's skin score. In the same embodiment, additional points will be added to a user's skin score based on the number of days since the user's first use of the skin cleanser. In one example, such points will be calculated as follows: 10 points will be added to a user's skin score if between 0 and 10 days have elapsed since first use; 20 points will be added to a user's skin score if between 10 and 30 days have elapsed since first use; 30 points will be added to a user's skin score if between 30 and 60 days have elapsed since first use; 40 points will be added to a user's skin score if over 60 days have elapsed since first use. Adding the value generated based on AMR with that generated based on number of days since first use will provide a skin score. Upon calculation of the same, a user's skin score will be communicated to the user on the display of a second device, such as a mobile phone. A skilled artisan will be able to determine how to select criteria for and how to generate skin scores according to a particular example based on various considerations, including the particular sensors of the skin cleanser and the data collected by the same. In other embodiments, any values collected with regard to days elapsed since first use and AMR may be used to calculate skin score. In different embodiments, uses per day, duration of use, other devices used in conjunction with the skin cleanser, and various other criteria may be factored wholly or in part into calculation of the skin score.

Based on the data received by a second device, such as a user's AMR, moisture type, skin age, and skin score, from the skin cleanser, the mobile phone application may recommend to the user any number of courses of action, including, but not limited to: using the skin cleanser more frequently, using the skin cleanser less frequently, using the skin cleanser for a longer period of time during an individual use, using the skin cleanser for a shorter period of time during an individual use, and using the skin cleanser on a particular area of the face and/or neck. Furthermore, the mobile phone may be able to recommend various creams, lotions, and/or beauty products to use that will enhance the user's skin based on the data collected. Of course, said applications or computer programs can be stored on any suitable electronic device; a mobile phone is not necessary for this process. A skilled artisan will be able to determine how best to integrate the skin cleanser with an application on another device according to a particular example based on various considerations, including the type of device to be integrated and the particular controller of the skin cleanser. In other embodiments, an application may suggest coating the exterior covering and other user-contacting portions of the skin cleanser with active ingredients, such as vitamin E, antioxidants, or silver nanoparticles. Additionally, with or without input from the application, the exterior covering can be coated with these ingredients by the user or pre-coated or can have a delivery mechanism in the skin cleanser that can store and provide these ingredients upon use in other embodiments.

Additional features may also be included in the skin cleanser in other embodiments. In a further embodiment, the skin cleanser includes a dosing mechanism integrated in the body of the device to dispense liquids or solid suspensions, such as for the delivery of silver nanoparticles, Vitamin E, etc. as described above. The dosing mechanism may include a reservoir, for example at the base of the skin cleanser, and a pump with an outlet to dispense contents of the reservoir to the textured surfaces or near the textured surfaces.

Another example optional feature that may be included with a particular skin cleanser, such as skin cleanser 2, is a security mechanism. In such an embodiment, the controller of the skin cleanser, such as controller 30, may include a scannable serial number. The serial number may be housed in the memory of the controller. After connecting with a second device, such as a personal computer, tablet, and/or mobile phone, the controller can send signals to the second device indicating the serial number. The connection may be made wirelessly or via wired connection.

The second device can then instruct the skin cleanser to perform one or more tests that only a skin cleanser having an authentic serial number will be able to complete. Such tests may include one or more of: a motor test, a battery test, and/or a moisture level test. If the skin cleanser is unable to perform these tests, the skin cleanser will be disabled from transmitting information to a second device.

If, however, the skin cleanser is able to perform said tests, the controller will be authorized to provide information pertaining to the device to the second device. Information that may be passed from the skin cleanser to the second device may include one or more of the following: the skin cleanser's Chip ID, Information pertaining to the battery of the skin cleanser, the Media Access Control (“MAC”) Address of the skin cleanser, and/or the serial number of the skin cleanser. This information may then be transmitted to the second device to another device, such as a personal computer, tablet, mobile phone, database, server, computer program, operating system, or other suitable target. The serial number of the controller the process associated with the same can help to provide software updates to the skin cleanser and/or confirm authenticity of a skin cleanser.

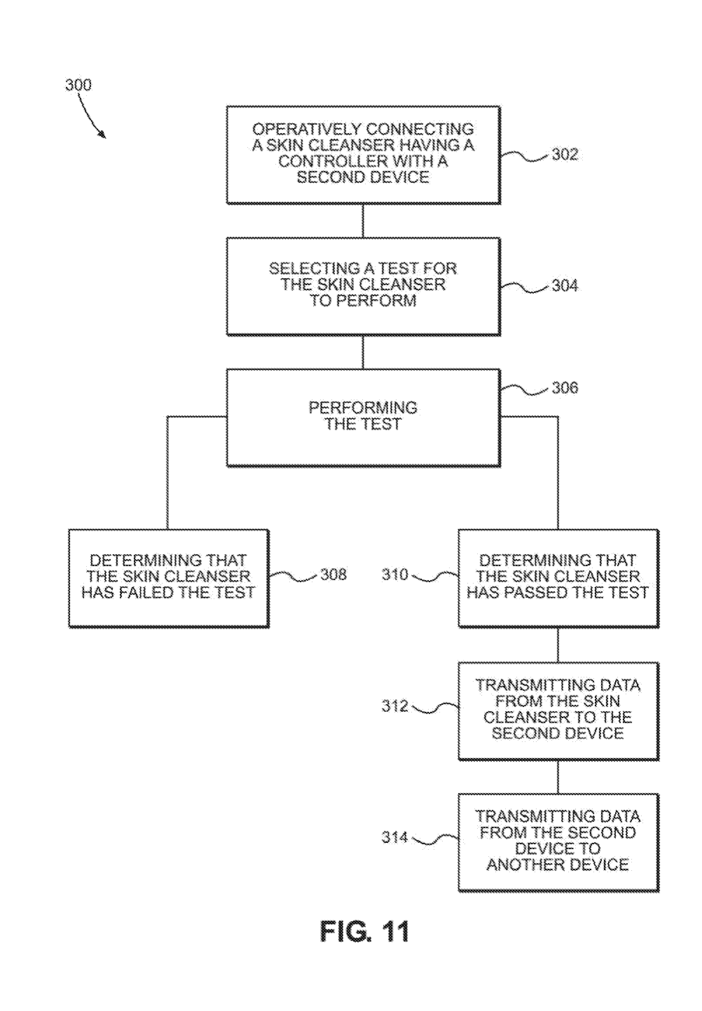

FIG. 11 is a flowchart representation of an example method 300 of verifying authenticity of a skin cleanser. Performance of the method results in the confirmation or denial of a particular skin cleanser's authenticity.

An initial step 302 comprises operatively connecting a skin cleanser having a controller, such as skin cleanser 2 having controller 30, with a second device such as a personal computer, tablet, and/or mobile phone, for example. Any suitable skin cleanser and/or controller may be used, however, in other embodiments. The connection may be made wirelessly or via a wired connection.

Another step 304 comprises the second device selecting a particular test for the skin cleanser to perform. Such tests may include one or more of: a motor test, a battery test, and/or a moisture level test in various embodiments. Any suitable test may be performed, though, in other embodiments.

Another step 306 comprises performing the test and/or test(s) selected in step 304.

Another step 308 comprises determining that the skin cleanser and/or its controller has failed the test(s) performed in step 306. In such an instance, the skin cleanser will be prevented from sending information pertaining to the skin cleanser to the second device. In other embodiments, the skin cleanser may be temporarily or permanently disabled after failure to perform the test(s).

Another step 310 comprises determining that the skin cleanser and/or its controller 30 has passed the test(s) performed in step 306.

Another step 312 comprises transmitting data from the skin cleanser to the second device. The data transmitted generally contains information pertaining to the skin cleanser and may include one or more of the following: the skin cleanser's Chip ID, Information pertaining to the battery of the skin cleanser, the Media Access Control (“MAC”) Address of the skin cleanser, and/or the serial number of the skin cleanser. Any pertinent information may be passed to the second device, however, in various embodiments.

Optionally, another step 314 comprises transmitting data from the second device to an additional device. The additional device may include one or more of: such as a personal computer, tablet, mobile phone, database, server, or other similar device. Any suitable additional device may be selected, however.

It is noted that it is advantageous to complete this method 300 in the order illustrated and described. However, any order is considered suitable.

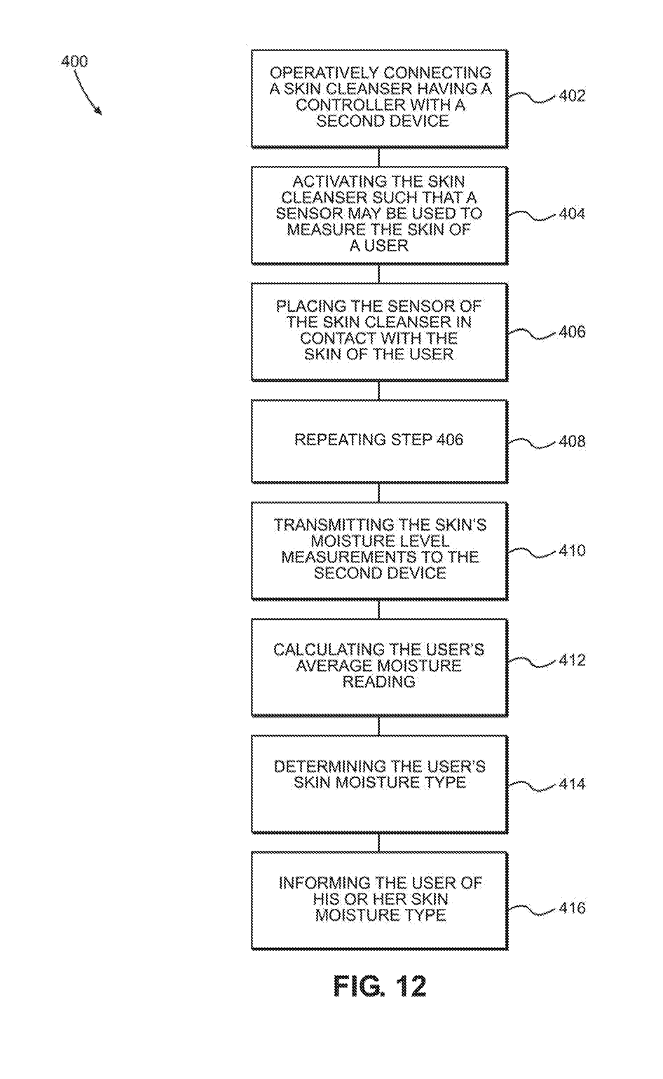

FIG. 12 is a flowchart representation of an example method 400 of calculating a user's skin moisture type. Performance of the method results in the calculation of a user's skin moisture type and providing said information to the user.

An initial step 402 comprises operatively connecting a skin cleanser having a controller, such as skin cleanser 2 having controller 30, with a second device such as a personal computer, tablet, and/or mobile phone, for example. Any suitable skin cleanser and/or controller may be used, however, in other embodiments. The connection may be made wirelessly or via a wired connection.

Another step 404 comprises activating the skin cleanser such that the sensor may be used to measure the moisture level of the skin of the user.

Another step 406 comprises placing the sensor of the skin cleanser in contact with the skin of the user such that it may measure the moisture level of the area of skin with which it is in contact. In various embodiments, the sensor may be placed in contact with one or more of the right cheek, left cheek, forehead, chin, and/or nose; it may also be placed in contact with various other portions of the face and/or neck. In additional embodiments, it may be placed in contact with said portions for any unit of time between one and sixty seconds in various embodiments.

Optionally, another step 408 comprises repeating step 406.

Another step 410 comprises transmitting the skin's moisture level readings and/or data to the second device. In various embodiments, the second device may comprise a mobile phone, a computer, a tablet, and/or a smart watch.

Another step 412 comprises the second device calculating the user's Average Moisture Reading (“AMR”). This is done via dividing the sum of the various moisture level readings by the total number of moisture level readings (as collected in step 406).

Another step 414 comprises determining the user's skin moisture type. Such a calculation will include the comparison of the AMR to a set of data relating to skin moisture levels stored by the second device on software (such as, for example, an application on a mobile phone). In the following example, the AMR can have a value between 0 and 3. The AMR is directly used to calculate a user's moisture type. A user having an AMR that is greater than 0 and less than or equal to 1.1 indicates that a user's skin has a low moisture level. An AMR that is greater than 1.1 and less than or equal to 1.35 indicates that a user's skin has a normal moisture level. An AMR that is greater than 1.35 and up to or equal to 3 indicates that a user's skin has a high moisture level. In various other examples, however, AMR may be calculated in some other manner or may have a range that extends below zero and/or above 3. In various examples, the AMR's reading may be used in a different manner to calculate moisture type. In some examples a user having an AMR of between about 0 and about 2, between about 0 and about 1, or between about 0 and about 0.5 may have a low moisture level. In other examples a user having an AMR of between about 0.5 and about 2, between about 1 and about 1.5, or between about 1.2 and about 1.3 may have a normal moisture level. In additional examples a user having an AMR of between about 1 and about 3, between about 1.3 and about 2.7, or between about 1.5 and about 3 may have a high moisture level.

Another step 416 comprises informing the user of his or her skin moisture type. The AMR and whether the user has low, normal, or high moisture levels (i.e., the user's moisture type) will be presented to the user on the display and/or screen of the second device.

It is noted that it is advantageous to complete this method 400 in the order illustrated and described. However, any order is considered suitable.

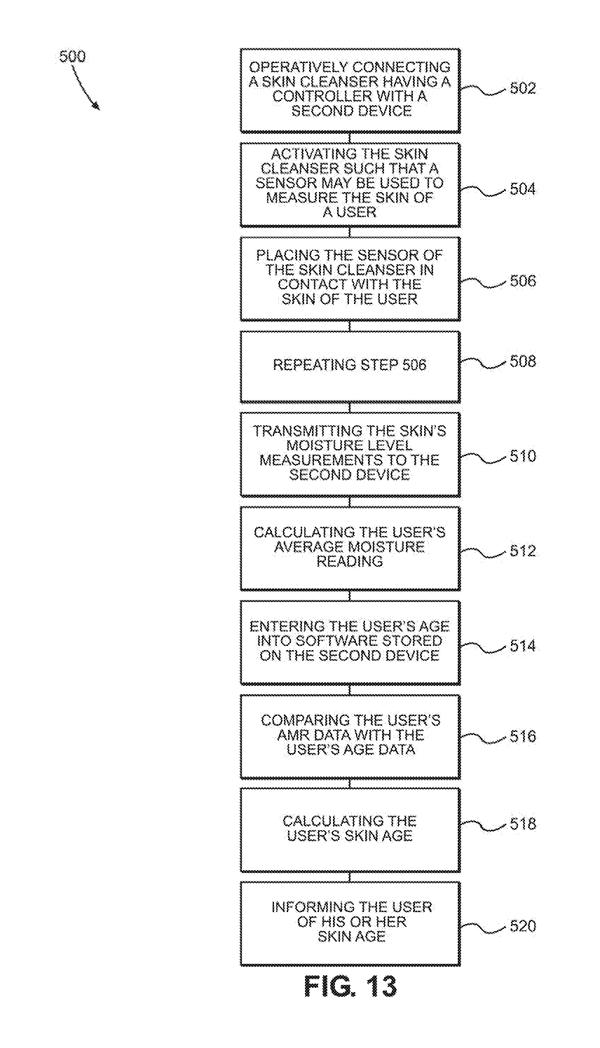

FIG. 13 is a flowchart representation of an example method 500 of calculating a user's skin age. Performance of the method results in the calculation of a user's skin age and providing said information to the user.

An initial step 502 comprises operatively connecting a skin cleanser having a controller, such as skin cleanser 2 having controller 30, with a second device such as a personal computer, tablet, and/or mobile phone, for example. Any suitable skin cleanser and/or controller may be used, however, in other embodiments. The connection may be made wirelessly or via a wired connection.

Another step 504 comprises activating the skin cleanser such that the sensor may be used to measure the moisture level of the skin of the user.

Another step 506 comprises placing the sensor of the skin cleanser in contact with the skin of the user such that it may measure the moisture level of the area of skin with which it is in contact. In various embodiments, the sensor may be placed in contact with one or more of the right cheek, left cheek, forehead, and/or nose; it may also be placed in contact with various other portions of the face and/or neck. In additional embodiments, it may be placed in contact with said portions for any unit of time between one and sixty seconds in various embodiments.

Optionally, another step 508 comprises repeating step 506.

Another step 510 comprises transmitting the skin's moisture level readings and/or data to the second device. In various embodiments, the second device may comprise a mobile phone, a computer, a tablet, and/or a smart watch.

Another step 512 comprises the second device calculating a user's Average Moisture Reading (“AMR”). This is done via dividing the sum of the various moisture level readings by the total number of moisture level readings (as collected in step 506).

Another step 514 comprises the user entering his or her age into software stored on the second device. For example, the user may enter his or her age into an application stored on a mobile phone.

Another step 516 comprises comparing the user's AMR data with the user's age data.

Another step 518 comprises calculating the user's skin age. Once the second device has received and stored the user's age and his or her AMR, it shall compare the same to determine skin age. In the following example, if the user's AMR is between 0 and 1.05, the user's skin age will be the user's age plus two. Accordingly, for example, a user that is 50 years old with an AMR of 0.9 will have a skin age of 52. If the user's AMR is greater than 1.05 and less than or equal to 1.1, the user's skin age will be the user's age plus one. Accordingly, for example, a user that is 50 years old with an AMR of 1.07 will have a skin age of 51. If the user's AMR is greater than 1.1 and less than or equal to 1.35, the user's skin age will be the user's age. Accordingly, for example, a user that is 50 years old with an AMR of 1.22 will have a skin age of 50. If the user's AMR is greater than 1.35 and less than or equal to 1.6, the user's skin age will be the user's age minus one. Accordingly, for example, a user that is 50 years old with an AMR of 1.42 will have a skin age of 49. If the user's AMR is greater than 1.6 and less than or equal to 3, the user's skin age will be the user's age minus two. Accordingly, for example, a user that is 50 years old with an AMR of 1.84 will have a skin age of 48. A skilled artisan will be able to determine how best to use AMR and a user's age to calculate skin age according to a particular example based on various considerations, including the characteristics of the anticipated user, the sensitivity of the sensors used, and the anticipated moisture of an area in which users reside. In various other examples, zero, one, two, three, four, or more than four years may be subtracted from a user's age based on various AMRs to determine skin age. In other examples, zero, one, two, three, four, or more than four years may be added to a user's age based on various AMR.

Another step 520 comprises informing the user of his or her skin age. This will be presented to the user on the display of the second device.

It is noted that it is advantageous to complete this method 500 in the order illustrated and described. However, any order is considered suitable.

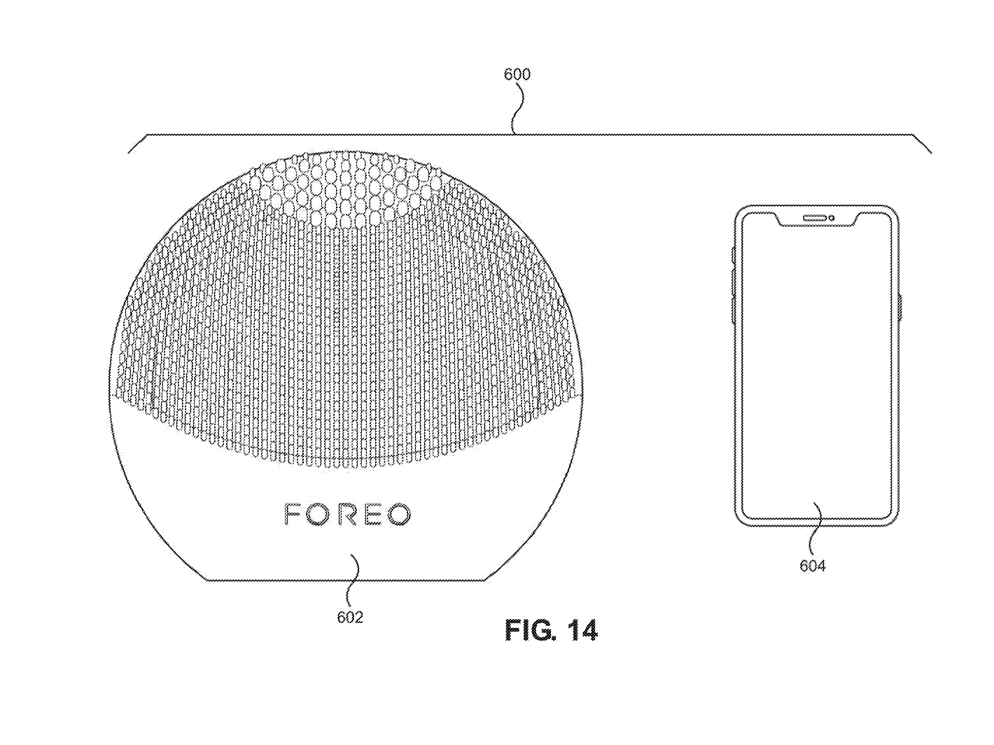

FIG. 14 illustrates a system 600 comprising a skin cleanser 602 and a second device 604. In the illustrated embodiment, skin cleanser 602 is identical to skin cleanser 2 and the second device 604 comprises a mobile phone. Accordingly, the skin cleanser 602 illustrated in this embodiment will operate in the same manner as skin cleanser 2 and the second device 604 will operate in the same manner as various mobile phones described above. The skin cleanser 602 will be operatively connected with the second device 604 such that it may transmit data regarding a use of the skin cleanser 602. Such data may include all data described above, including but not limited to, number of uses, AMR, duration of average use, time of use, and where a user uses the device. The second device 604 will then use said data in calculating, generating, and/or displaying various graphics, data, pieces of information, and/or other similar content, as described above. The second device 604, for example, may calculate AMR, skin moisture type, skin age, and/or skin score, among other possible examples.

In all examples, a skin cleanser and its various components may be formed of any suitable material, including presently known and later-developed materials. A skilled artisan will be able to determine appropriate materials for an example skin cleanser based on various considerations, including the size and shape of the skin cleanser, the motor housed within the skin cleanser, and any sensors utilized in conjunction with the skin cleanser.

Those with ordinary skill in the art will appreciate that various modifications and alternatives for the described and illustrated embodiments can be developed in light of the overall teachings of the disclosure. Accordingly, the particular arrangements disclosed are intended to be illustrative only and not limiting as to the scope of the invention, which is to be given the full breadth of the appended claims and any and all equivalents thereof.

What is claimed is:

1. A skin cleanser having a skin cleanser body and configured to be used on skin of a user, comprising:
a substantially flat base configured to stand unaided on a substantially flat surface, said skin cleanser body having a cross-sectional shape that is longer in a first direction substantially parallel to the base than in a second direction substantially parallel to the base;
a sensor disposed within said skin cleanser body, the sensor having a first sensor end and a second sensor end extending outside said skin cleanser body, the sensor configured to measure moisture level readings of said skin;
a controller disposed within said skin cleanser body, the controller being capable of collecting, storing, and transmitting data related to the moisture level readings;
a silicone exterior covering that covers substantially all of an exterior of said skin cleanser body, the silicone exterior covering having a first side and a second side, the first side defining a first textured surface area that is integrally formed with the silicone exterior covering, the first textured surface area comprising a first set of touch-points, the second side defining passageways through which the first sensor end and the second sensor end;
a motor disposed within said skin cleanser body configured to produce pulsations of the skin cleanser; and
at least one control disposed on said skin cleanser body configured to operate the motor.

2. The skin cleanser of claim 1, wherein the sensor is configured to transmit moisture level readings to the controller.

3. The skin cleanser of claim 2, wherein the moisture level readings are determined by measuring the voltage differential between a first resistor and a second resistor, the second resistor being configured to decrease when the sensor contacts or is in close proximity to said skin of said user.

4. The skin cleanser of claim 3, wherein the controller transmits the data relating to the moisture level readings to a mobile phone having a software application configured to collect and analyze the moisture level readings.

5. The skin cleanser of claim 4, wherein the mobile phone calculates an Average Moisture Reading (“AMR”) based on multiple moisture level readings.

6. The skin cleanser of claim 5, wherein the AMR is measured in volts.

7. The skin cleanser of claim 6, wherein the AMR is used to calculate said user's skin moisture type;
wherein an AMR of greater than 0 volts and less than or equal to 1.1 volts indicates said skin has a low moisture skin type;
wherein an AMR of greater than 1.1 volts and less than or equal to 1.35 volts indicates said skin has a normal moisture skin type; and
wherein an AMR of greater than 1.35 volts indicates said skin has a high moisture skin type.

8. The skin cleanser of claim 7, wherein the mobile phone includes a display on which said user's AMR and skin moisture type are displayed.

9. The skin cleanser of claim 6, wherein the software application is configured to collect and store the age of said user.

10. The skin cleanser of claim 9, wherein the AMR and age of said user are used to calculate said user's skin age;
wherein if the AMR is greater than 0 volts and less than or equal to 1.05 volts, said user's skin age is said user's age plus two;
wherein if the AMR is greater than 1.05 volts and less than or equal to 1.1 volts, said user's skin age is said user's age plus one;
wherein if the AMR is greater than 1.1 volts and less than or equal to 1.35 volts, said user's skin age is said user's age;
wherein if the AMR is greater than 1.35 volts and less than or equal to 1.6 volts, said user's skin age is the user's age minus one; and
wherein if the AMR is greater than 1.6 volts, said user's skin age is said user's age minus two.

11. The skin cleanser of claim 10, wherein the mobile phone includes a display on which said user's AMR and skin age are displayed.

12. The skin cleanser of claim 1, wherein the sensor comprises a capacitive sensor.

13. The skin cleanser of claim 1, wherein the first sensor end and second sensor end are gold-plated.

14. A skin cleanser having a skin cleanser body and configured to be used on skin of a user, comprising:
a substantially flat base configured to stand unaided on a substantially flat surface, said skin cleanser body having a cross-sectional shape that is longer in a first direction substantially parallel to the base than in a second direction substantially parallel to the base;
a sensor disposed within said skin cleanser body, the sensor having a first sensor end and a second sensor end extending outside said skin cleanser body, the first sensor end and second sensor end being gold-plated, the sensor configured to measure moisture level readings of said skin;
a controller disposed within said skin cleanser body, the controller being capable of collecting, storing, and transmitting data related to the moisture level readings of said skin;
a silicone exterior covering that covers substantially all of an exterior of said skin cleanser body, the silicone exterior covering having a first side and a second side, the first side defining a first textured surface area that is integrally formed with the silicone exterior covering, the first textured surface area comprising a first set of touch-points, the second side defining passageways through which the first sensor end and the second sensor end extend;
a motor disposed within said skin cleanser body configured to produce pulsations of the skin cleanser; and
at least one control disposed on said skin cleanser body configured to operate the motor.

15. The skin cleanser of claim 14, wherein the first side defines a second textured surface area that is integrally formed with the silicone exterior covering, the second textured surface area comprising a second set of touch-points.

16. The skin cleanser of claim 15, wherein the second side of the silicone exterior covering defines a decorative design.

17. The skin cleanser of claim 14, wherein the moisture level readings are determined by measuring the voltage differential between a first resistor and a second resistor, the second resistor being configured to decrease when the sensor contacts or is in close proximity to said skin of said user.

18. A method of calculating a skin age of skin of a user, comprising the steps of:
operatively connecting a skin cleanser with a mobile phone, the skin cleanser having a skin cleanser body and configured to be used on the skin of a user, comprising:
a substantially flat base configured to stand unaided on a substantially flat surface, said skin cleanser body having a cross-sectional shape that is longer in a first direction substantially parallel to the base than in a second direction substantially parallel to the base;
a sensor disposed within said skin cleanser body, the sensor having a first sensor end and a second sensor end extending outside said skin cleanser body, the first sensor end and the second sensor end being gold-plated, the sensor configured to measure moisture level readings of said skin;
a controller disposed within said skin cleanser body, the controller being capable of collecting, storing, and transmitting data related to the moisture level readings of said skin;
a silicone exterior covering that covers substantially all of an exterior of said skin cleanser body, the silicone exterior covering having a first side and a second side, the first side defining a first textured surface area that is integrally formed with the silicone exterior covering, the first textured surface area comprising a first set of touch-points, the second side defining passageways through which the first sensor end and the second sensor end extend;
a motor disposed within said skin cleanser body configured to produce pulsations of the skin cleanser; and
at least one control disposed on said skin cleanser body configured to operate the motor;
activating the skin cleanser such that the sensor measures moisture level readings of said skin of said user;
placing the sensor in contact with said skin of said user in order to allow the sensor to measure the moisture level readings;
transmitting the moisture level readings to the mobile phone;
calculating said user's AMR via software stored on the mobile phone;
entering a user's age into the software stored on the mobile phone;
comparing the user's AMR data with said user's age; and
calculating said user's skin age.

19. The method of claim 18, wherein if the AMR is greater than 0 volts and less than or equal to 1.05 volts, said user's skin age is said user's age plus two;
wherein if the AMR is greater than 1.05 volts and less than or equal to 1.1 volts, said user's skin age is said user's age plus one;
wherein if the AMR is greater than 1.1 volts and less than or equal to 1.35 volts, said user's skin age is said user's age;
wherein if the AMR is greater than 1.35 volts and less than or equal to 1.6 volts, said user's skin age is said user's age minus one; and
wherein if the AMR is greater than 1.6 volts, said user's skin age is said user's age minus two.

20. The method of claim 19, wherein the AMR is calculated by measuring the voltage differential between a first resistor and a second resistor, the second resistor being configured to decrease when the sensor contacts or is in close proximity to said skin of said user.
